(19) United States
(12) Patent Application Publication
(10) Pub. No.: US 2002/0142043 A1
(43) Pub. Date: Oct. 3, 2002

(54) POWDER CONTAINING FAT-SOLUBLE DRUG

(76) Inventors: YOSHITERU KATO, SAITAMA PREFECTURE (JP); YUKEI NAKAMURA, SAITAMA PREFECTURE (JP); KIMIO TATEISHI, SAITAMA PREFECTURE (JP); SHINICHIRO YOKOI, TOYAMA PREFECTURE (JP); NOBUKAZU TANAKA, TOYAMA PREFECTURE (JP); TERUMASA HOSOKAWA, TOYAMA PREFECTURE (JP)

Correspondence Address: WENDEROTH LIND & PONACK, 2033 K STREET NW, SUITE 800, WASHINGTON, DC, 20006, US

(21) Appl. No.: 09/463,273

(22) PCT Filed: May 24, 1999
(86) PCT No.: PCT/JP99/02705

(30) Foreign Application Priority Data
May 26, 1998 (JP) 144451/1998
May 19, 1999 (JP) 174446/1999

(51) Int. Cl. A61K 9/00; A61K 9/14

(52) U.S. Cl. 424/489; 424/400

(57) ABSTRACT

Flowable powder comprising (A) a fat-soluble drug, (B) gelatin and/or casein, and (C) an adsorbent, and optionally (D) a water-soluble polymer, and a production process thereof.

### APPLICATION FIELD IN INDUSTRY

The present invention relates to powder containing a fat-soluble drug and having good flowability, and a production process thereof.

A great number of powder containing a fat-soluble vitamin or production processes thereof have been known. For example, U.S. Pat. No. 2,756,177 discloses a production process of powder obtained by drying an emulsion containing a vitamin active substance, water, gelatin and/or gum arabic, and a saccharide. Japanese Patent Application Laid-Open No. 59915/1983 discloses a vitamin E-containing powder preparation comprising 50 to 60 wt. % of vitamin E, 0.5 to 2.0 wt. % of silicon dioxide, 1 to 25 wt. % of hydrolyzed gelatin and 20 to 30 wt. % of sodium casein, and Japanese Patent Application Laid-Open No. 200273/1993 discloses a powder product obtained by dispersing a core substance in an aqueous solution of amylose and spraying the dispersion using hydrophobic silica as an auxiliary. Silicon dioxide and hydrophobic silica in these techniques are used for imparting flowability to the respective resulting powders.

On the other hand, Japanese Patent Application Laid-Open No. 501686/1991 (through PCT route) discloses a process for producing free-flowing, spray-dried edible powder containing an edible oil by using hydrolyzed gelatin having a weight average molecular weight of about 15,000 to about 35,000 as measured by gel permeation chromatography.

### PROBLEMS SOUGHT FOR SOLUTION BY THE INVENTION

Such many techniques as described above have been known for obtaining flowable powder containing a fat-soluble drug such as vitamin E. However, there is a demand for development of more improved techniques from the viewpoints of enhancing the content of the fat-soluble drug as high as possible and preventing the exudation of the fat-soluble drug and its sticking to a tableting machine. In addition, hydrolyzed gelatin, amylose and the like are comparatively expensive, and so improvement is required from the viewpoint of cost. The present inventors have carried out an extensive investigation with a view toward solving these problems. As a result, it has been found that the problems can be solved by the following means, thus leading to completion of the present invention.

### MEANS FOR THE SOLUTION OF THE PROBLEMS

The present invention relates to flowable powder comprising (A) a fat-soluble drug, (B) at least one selected from the group consisting of gelatin and casein, and (C) an adsorbent. The present invention also relates to flowable powder comprising (A) a fat-soluble drug, (B) at least one selected from the group consisting of gelatin and casein, (D) a water-soluble polymer, and (C) an adsorbent. The present invention further relates to a process for producing flowable powder, which comprises emulsifying and dispersing (A) a fat-soluble drug, (B) at least one selected from the group consisting of gelatin and casein, and (C) an adsorbent, and optionally (D) a water-soluble polymer in water and spray-drying the resultant dispersion.

The term “fat-soluble drug” as used in the present invention means a physiologically active substance insoluble or hardly soluble in water and relatively easy to dissolve in alcohols such as octanol, oils and fats, and the like. The melting point of the fat-soluble drug is desirably about 80° C. or lower. As examples of the fat-soluble drug, may be mentioned vitamin E family and the like. More specifically, there may be mentioned dl-α-tocopherol, d-α-tocopherol, dl-α-tocopherol acetate, d-α-tocopherol acetate, and besides β, γ and δ homologues thereof, dl-α-tocopherol succinate, d-α-tocopherol succinate, vitamin A oil, tocotrienol, etc. Besides the above, drugs such as teprenone, indomethacin farnesl, tocopherol nicotinate, ubidecarenone, menatetrenone and phytonadione may also be used.

In the present invention, no particular limitation is imposed on gelatin. However, it generally means that produced by treating bone, skin, ligamentum, tendon or the like of an animal with an acid or alkali and extracting the thus-obtained collagen with water under heating. No particular limitation is imposed on the isotonic point, molecular weight, viscosity and the like of gelatin used in the present invention. With respect to gelatin wherein the molecular weight thereof has been controlled to thousands to tens of thousands by hydrolysis, for example, such a gelatin is comparatively expensive, and hence, it has a great effect from an economical point of view to use gelatin which is not subjected to such a treatment.

In the present invention, no particular limitation is imposed on casein. Casein is a phosphoprotein which is a main component of milk, and is not a single substance, but a mixture of similar proteins. It consists of at least three components (α, β and γ), and its molecular weight is about 75,000 to 375,000. In the present invention, the use of sodium casein having good solubility is preferred because of its good handling property.

In the present invention, the adsorbent means powder of a porous inorganic substance. As examples thereof, may be mentioned silicon oxide, calcium silicate, magnesium alumino silicate [Neusilin (trademark) A, product of Fuji Chemical Industry Co., Ltd.], magnesium alumino metasilicate [Neusilin (trademark), product of Fuji Chemical Industry Co., Ltd.], etc. Silicon dioxide, calcium silicate and magnesium altimino metasilicate are preferred. In the present invention, a component having good oil-absorbing ability, such as dextrin or calcium hydrogenphosphate, may be used in combination with the adsorbent if desired.

In the present invention, the water-soluble polymer is generally a cellulose derivative or polyvinyl alcohol. As examples of the cellulose derivative, may be mentioned hydroxypropyl cellulose, hydroxypropyl methylcellulose and sodium carboxymethylcellulose, and the like. With respect to these cellulose derivatives, some kinds of derivatives have been known according to properties such as viscosity. However, no particular limitation is imposed thereon in the present invention. When the water-soluble polymer is used in the present invention, the emulsion stability of the fat-soluble drug, gelatin, casein and adsorbent in water is improved, so that the quality of the resulting flowable powder is enhanced.

Examples of cellulose and/or cellulose derivatives include those that are soluble in water or dissolve in water to become viscous liquids. Examples thereof include methylcellulose, hydroxypropyl cellulose [for example, Nisso HPC, trade name: product of Nippon Soda Co., Ltd.; and Shinetsu HPC, trade name; product of Shi-Etsu Chemical Co., Ltd.], hydroxypropyl methylcellulose-2208, -2906 and -2910 [for example, Metholose 90SH, 65SH and 60SH, trade names: products of Shi-Etsu Chemical Co., Ltd.; and Methocel K, F and E, trade names, and Marbolose, trade name TC-51:products of Dow Chemical Co., Ltd.], and hydroxyethyl cellulose, and the like.

As cellulose and/or cellulose derivatives that are insoluble in water or partially dissolve in or swell with water, there may also be used, for example, crystalline cellulose, sodium crystalline cellulose.carmelose, ethyl cellulose, hydroxypropyl cellulose having a low degree of substitution, hydroxypropyl methylcellulose phthalate, hydroxypropyl methylcellulose acetate succinate, carmelose, calcium carmellose, sodium carmellose, sodium crosscarmellose, carboxymethylethyl cellulose and cellulose acetate phthalate, and the like.

The proportions of the fat-soluble drug, gelatin and/or casein, and adsorbent in the present invention are generally 0.05 to 0.5 parts by weight for gelatin and/or casein, and 0.1 to 0.8 parts by weight for the adsorbent, per part by weight of the fat-soluble drug, and preferably 0.05 to 0.25 parts by weight for gelatin and/or casein, and 0.2 to 0.7 parts by weight for the adsorbent, per part by weight of the fat-soluble drug. When the water-soluble polymer is further used in the present invention, the proportions of these components are generally 0.05 to 0.5 parts by weight for gelatin and/or casein, 0.1 to 0.8 parts by weight for the adsorbent, and 0.01 to 0.1 parts by weight for the water-soluble polymer, per part by weight of the fat-soluble drug, and preferably 0.05 to 0.25 parts by weight for gelatin and/or casein, 0.2 to 0.7 parts by weight for the adsorbent, and 0.005 to 0.1 parts by weight, more preferably 0.015 to 0.1 parts by weight for the water-soluble polymer, per part by weight of the fat-soluble drug.

No particular limitation is imposed on the production process of the flowable powder in the present invention. The flowable powder can be produced, for example, in accordance with the following process. However, for convenience' sake, description will be given as to a case where gelatin is used.

Gelatin is dissolved in purified water heated to about 50 to 60° C., and a water-soluble polymer such as hydroxypropyl methylcellulose is further added and dissolved if circumstances require. Vitamin E (dl-αtocopherol acetate) is then added to the solution, and the resultant mixture is stirred and emulsified for about 15 minutes by means of a high-speed stirring machine, for example, a Homomixer (trade name) or the like. Additional water heated to about 50 to about 60° C. is added as needed, and an adsorbent such as calcium silicate is added to the solution to stir the resultant mixture for about 15 minutes, thereby obtaining a homogeneous suspension. The viscosity of the thus-obtained suspension is generally 20 to 1,000 CP, preferably 30 to 1,000 CP. The suspension is spray-dried by a spray dryer, whereby flowable powder containing vitamin E can be obtained. As the spray dryer, may be used any machine of the disk type and the nozzle type. Conditions of the spray drying are those generally used. For example, the inlet temperature is 210° C., and the outlet temperature is 130° C.

The particle diameter of the flowable powder obtained by the present invention is generally 0.05 to 0.5 mm, and the angle of repose thereof is generally 30 to 40°, so that the powder has good flowability.

When the melting point of the fat-soluble drug used is high, for example, d-α-tocopherol succinate having a melting point of about 75° C. is used, a homogeneous suspension can be obtained by raising the temperature at the time the solution is prepared to about 80° C. to conduct stirring. In this case, the suspension must not be always heated upon spraying once the homogeneous suspension is prepared.

### EFFECTS OF THE INVENTION

According to the present invention, there can be produced flowable powder containing a fat-soluble drug at a high content of 50% or higher. The flowable powder according to the present invention can be prepared by a direct tableting process into tablets without causing the exudation of the fat-soluble drug and its sticking to a tableting machine by mixing it with an excipient such as a saccharide, or cellulose or a derivative thereof. A marked effect of the present invention is that stain or smear on a spray dryer is extremely slight. When powder containing a fat-soluble drug has heretofore been produced, cleaning has been extremely troublesome because the drug and the like have stuck to the spray dryer. However, cleaning and the like are extremely easy because the drug and the like scarcely stick after the powder according to the present invention is produced.

According to the present invention, there could be provided vitamin E-containing powder which scarcely undergoes the exudation and decomposition of the vitamin E even when the powder is stored for a long period of time, and hence has high shelf stability; the vitamin E is not degraded upon the production thereof The vitamin E-containing powder according to the present invention has been powder which is free from the exudation of a vitamin E component out of the surface of the powder, is dry, undergoes no aggregation among particles and is easy to store, quantify and transport. According to the present invention, a recovery rate is markedly improved because the powder does not stick to the inner wall of a spry dryer upon the production thereof. In addition, since the spray drying treatment can be conducted in a temperature range comparatively higher than that in the conventional processes, the time required for the drying treatment is shortened, and moreover the time during which the powder is exposed to heat is shortened. Therefore, the production process of the present invention is extremely efficient.

The vitamin E-containing powder according to the present invention is useful as an antioxidant and stabilizer for food, and a medicament.

### EXAMPLES

The present invention will hereinafter be described in more detail by the following Examples. However, the present invention is not limited to these examples.

### Example 1

Dissolved in 200 g of purified water heated to 50° C. were 10 g of gelatin, and 120 g of dl-α-tocopherol acetate were added to the solution. The resultant mixture was stirred for 10 minutes by a Homomixer (trade name). Further, 200 g of purified water heated to about 50° C. were added to the mixture, followed by stirring for 10 minutes by the Homomixer. To the resultant mixture were added 70 g of calcium silicate (product of Tokuyama Soda Co., Ltd., trade name: Florite RE), and the resultant mixture was stirred for 15 minutes by the Homomixer to obtain a homogeneous suspension. The viscosity of the suspension was 150 cp. The suspension was spray-dried under the following conditions to obtain 70 g of flowable powder. The resultant powder had good flowability, a particle diameter of 0.07 mm and a dl-α-tocopherol acetate content of 590 mg per gram of the powder.

Spray drying conditions: inlet temperature 200° C., outlet temperature 100° C., disk revolution speed 30,000 rpm, and spray speed 1.3 L/hr.

### Example 2

Dissolved in 200 g of purified water heated to 50° C. were 20 g of gelatin, and 120 g of dl-α-tocopherol acetate were added to the solution. The resultant mixture was stirred for 10 minutes by a Homomixer (trade name). Further, 200 g of purified water heated to about 50° C. were added to the mixture, followed by stirring for 10 minutes by the Homomixer. To the resultant mixture were added 30 g of calcium silicate (product of Tokuyama Soda Co., Ltd., trade name: Florite RE), and the resultant mixture was stirred for 15 minutes by the Homomixer to obtain a homogeneous suspension. The viscosity of the suspension was 300 cp. The suspension was spray-dried under the following conditions to obtain 60 g of flowable powder. The resultant powder had good flowability, a particle diameter of 0.07 mm and a dl-α-tocopherol acetate content of 700 mg per gram of the powder.

Spray drying conditions: inlet temperature 200° C., outlet temperature 100° C., disk revolution speed 30,000 rpm, and spray speed 1.5 L/hr.

### Example 3

Dissolved in 260 g of purified water heated to 50° C. were 20 g of gelatin and 10 g of hydroxypropyl methylcellulose (product of Shin-Etsu Chemical Co., Ltd., trade name: TC-5E), and 120 g of dl-α-tocopherol acetate were added to the solution. The resultant mixture was stirred for 10 minutes by a Homomixer (trade name). Further, 200 g of purified water heated to about 50° C. were added to the mixture, followed by stirring for 10 minutes by the Homomixer. To the resultant mixture were added 30 g of calcium silicate (product of Tokuyama Soda Co., Ltd., trade name: Florite RE), and the resultant mixture was stirred for 15 minutes by the Homomixer to obtain a homogeneous suspension. The viscosity of the suspension was 300 cp. The suspension was spray-dried under the following conditions to obtain 120 g of flowable powder. The resultant powder had good flowability, a particle diameter of 0.07 mm and a dl-α-tocopherol acetate content of 660 mg per gram of the powder.

Spray drying conditions: inlet temperature 200° C., outlet temperature 100° C., disk revolution speed 30,000 rpm, and spray speed 1.5 L/hr.

### Example 4

Flowable powder was obtained in the same manner as in Example 3 except that 120 g of dl-α-tocopherol acetate were changed to 120 g of d-α-tocopherol acetate. The viscosity of the suspension was 350 cp. The suspension was spray-dried under the following conditions to obtain 110 g of flowable powder. The resultant powder had good flowability, a particle diameter of 0.07 mm and a dl-α-tocopherol acetate content of 658 mg per gram of the powder.

Spray drying conditions: inlet temperature 200° C., outlet temperature 100° C., disk revolution speed 30,000 rpm, and spray speed 1.5 L/hr.

### Example 5

Dissolved in 260 g of purified water heated to 50° C. were 30 g of gelatin and 10 g of hydroxypropyl methylcellulose product of Shin-Etsu Chemical Co., Ltd., trade name: TC-5E), and 120 g of d-α-tocopherol acetate were added to the solution. The resultant mixture was stirred for 10 minutes by a Homomixer (trade name). Further, 300 g of purified water heated to about 50° C. were added to the mixture, followed by stirring for 10 minutes by the Homomixer. To the resultant mixture were added 35 g of calcium silicate (product of Tokuyama Soda Co., Ltd., trade name: Florite RE), and the resultant mixture was stirred for 15 minutes by the Homomixer to obtain a homogeneous suspension. The viscosity of the suspension was 400 cp. The suspension was spray-dried under the following conditions to obtain 120 g of flowable powder. The resultant powder had good flowability, a particle diameter of 0.07 mm and a d-α-tocopherol acetate content of 610 mg per gram of the powder.

Spray drying conditions: inlet temperature 200° C., outlet temperature 100° C., disk revolution speed 30,000 rpm, and spray speed 1.5 L/hr.

### Example 6

Flowable powder was obtained in the same manner as in Example 1 except that calcium silicate was changed to anhydrous silicic acid (trade name: Sylysia). The viscosity of the suspension was 130 cp. The suspension was spray-dried under the following conditions to obtain 50 g of flowable powder. The resultant powder had good flowability, a particle diameter of 0.06 mm and a dl-α-tocopherol acetate content of 590 mg per gram of the powder.

Spray drying conditions: inlet temperature 200° C., outlet temperature 100° C., disk revolution speed 30,000 rpm, and spray speed 1.3 L/hr.

### Example 7

Flowable powder was obtained in the same manner as in Example 3 except that 10 g of hydroxypropylmethyl cellulose (trade name: TC-5E) were changed to 2 g of hydroxypropylmethyl cellulose (trade name: TC-5R). The viscosity of the suspension was 200 cp. The suspension was spray-dried under the following conditions to obtain 115 g of flowable powder. The resultant powder had good flowability, a particle diameter of 0.07 mm and a dl-α-tocopherol acetate content of 698 mg per gram of the powder.

Spray drying conditions: inlet temperature 200° C., outlet temperature 100° C., disk revolution speed 30,000 rpm, and spray speed 1.5 L/hr.

### Example 8

Flowable powder was obtained in the same manner as in Example 3 except that 10 g of hydroxypropylmethyl cellulose (trade name: TC-5E) were changed to 1 g of polyvinyl alcohol. The viscosity of the suspension was 200 cp. The suspension was spray-dried under the following conditions to obtain 85 g of flowable powder. The resultant powder had good flowability, a particle diameter of 0.06 mm and a dl-α-tocopherol acetate content of 690 mg per gram of the powder.

Spray drying conditions: inlet temperature 200° C., outlet temperature 100° C., disk revolution speed 30,000 rpm, and spray speed 1.5 L/hr.

### Example 9

Dissolved in 62 kg g of purified water heated to 50° C. were 5.0 kg of gelatin and 2.5 kg of hydroxypropyl methylcellulose (product of Shin-Etsu Chemical Co., Ltd., trade name: TC-5R), and 30.0 kg of dl-α-tocopherol acetate were added to the solution. The resultant mixture was stirred for 10 minutes by a Homomixer (trade name). Further, 55 kg of purified water heated to about 50° C. were added to the mixture, followed by stirring for 15 minutes by the Homomixer. To the resultant mixture were added 7.5 kg of calcium silicate (product of Tokuyama Soda Co., Ltd., trade name: Florite RE), and the resultant mixture was stirred for 15 minutes by the Homomixer to obtain a homogeneous suspension. The viscosity of the suspension was 150 cp. The suspension was spray-dried by a spray dryer of the nozzle type having a water-evaporating capability of 250 kg/hr under the following conditions to obtain 40 kg of flowable powder. No spray trouble occurred during the operation, and the inner wall of the spray dryer after the spray drying was neither stuck nor stained with the sprayed solution, powder and the like, and can be easily cleaned with water, and so operability was extremely excellent. The resultant powder had good flowability, an angle of repose of 35°, a particle diameter of 0.19 mm and a dl-α-tocopherol acetate content of 661 mg per gram of the powder.

Spray drying conditions: inlet temperature 210° C., outlet temperature 130° C., nozzle bore 1.0 mm, and spray speed 100 kg/hr.

### Example 10

A process was carried out in the same manner as in Example 9 except that 2.5 kg of hydroxypropyl methylcellulose (trade name: TC-5E) were used in place of 2.5 kg of hydroxypropyl methylcellulose (trade name: TC-5R), and 30.0 kg of d-α-tocopherol acetate were used in place of 30.0 kg of dl-α-tocopherol acetate. The viscosity of the suspension was 90 cp. The suspension was spray-dried by a spray dryer of the nozzle type having a water-evaporating capability of 250 kg/hr under the following conditions to obtain 42 kg of flowable powder. No spray trouble occurred during the operation, and the inner wall of the spray dryer after the spray drying was neither stuck nor stained with the sprayed solution, powder and the like, and can be easily cleaned with water, and so operability was extremely excellent. The resultant powder had good flowability, an angle of repose of 34°, a particle diameter of 0.17 mm and a d-α-tocopherol acetate content of 659 mg per gram of the powder.

Spray drying conditions: inlet temperature 210° C., outlet temperature 130° C., nozzle bore 1.0 mm, and spray speed 100 kg/hr.

### Example 11

Dissolved in 200 g of purified water heated to 50° C. were 24 g of gelatin and 1.2 g of sodium carboxymethyl cellulose (the name of a maker: Shin-Etsu Chemical Co., Ltd.), and 120 g of dl-α-tocopherol acetate (vitamin E) were added to the solution. The resultant mixture was stirred for 10 minutes by a homomixer (manufactured by Tokushu Kika Kogyo Co., Ltd., trade name: T.K Homomixer). Further, 250 g of purified water heated to about 50° C. were added to the mixture, followed by stirring for 10 minutes by the homomixer. To the resultant mixture were added 34.8 g of calcium silicate, and the resultant mixture was stirred for 15 minutes by the homomixer to obtain a homogeneous suspension. The viscosity of the suspension was 108 cp. The suspension was spray-dried under the following conditions to obtain 116 g of flowable powder having a vitamin E content of 66.7%. The resultant powder had good flowability.

Spray drying conditions: inlet temperature 210° C., outlet temperature 105° C., disk revolution speed 30,000 rpm, and spray speed 2.0 L/hr.

### Example 12

Dissolved in 200 g of purified water heated to 50° C. were 24 g of gelatin and 1.2 g of sodium carboxymethyl cellulose (the name of a maker: Daicel Chemical Industries, Ltd.), and 120 g of dl-α-tocopherol acetate (vitamin E) were added to the solution. The resultant mixture was stirred for 10 minutes by a Homomixer (trade name). Further, 250 g of purified water heated to about 50° C. were added to the mixture, followed by stirring for 10 minutes by the Homomixer. To the resultant mixture were added 30 g of calcium silicate and 4.8 g of dextrin, and the resultant mixture was stirred for 15 minutes by the homomixer to obtain a homogeneous suspension. The viscosity of the suspension was 100 cp. The suspension was spray-dried under the following conditions to obtain 128 g of flowable powder having a vitamin E content of 66.7%. The resultant powder had good flowability. The bulk density of the powder was 3.24 ml/g.

Spray drying conditions: inlet temperature 220° C., outlet temperature 105° C., disk revolution speed 30,000 rpm, and spray speed 2.0 L/hr.

### Example 13

Dissolved in 180 g of purified water heated to 50° C. were 21 g of gelatin and 3.0 g of sodium carboxymethyl cellulose (the name of a maker: Daicel Chemical Industries, Ltd.), and 126 g of dl-α-tocopherol acetate (vitamin E) were added to the solution. The resultant mixture was stirred for 10 minutes by a Homomixer (trade name). Further, 250 g of purified water heated to about 50° C. were added to the mixture, followed by stirring for 10 minutes by the Homomixer. To the resultant mixture were added 30 g of calcium silicate, and the resultant mixture was stirred for 15 minutes by the homomixer to obtain a homogeneous suspension. The viscosity of the suspension was 129 cp. The suspension was spray-dried under the following conditions to obtain 120 g of flowable powder having a vitamin E content of 70%. The resultant powder had good flowability. The bulk density of the powder was 3.5 ml/g.

Spray drying conditions: inlet temperature 220° C., outlet temperature 105° C., disk revolution speed 30,000 rpm, and spray speed 2.0 L/hr.

### Example 14

Dissolved in 180 g of purified water heated to 50° C. were 21 g of gelatin and 3.0 g of sodium carboxymethyl cellulose (the name of a maker: Daicel Chemical Industries, Ltd.), and 126 g of dl-α-tocopherol acetate (vitamin E) were added to the solution. The resultant mixture was stirred for 10 minutes by a Homomixer (trade name). Further, 250 g of purified water heated to about 50° C. were added to the mixture, followed by stirring for 10 minutes by the Homomixer. To the resultant mixture were added 30 g of calcium silicate, and the resultant mixture was stirred for 15 minutes by the homomixer to obtain a homogeneous suspension. The viscosity of the suspension was 129 cp. The suspension was spray-dried under the following conditions to obtain 143 g (yield: 79.5%) of flowable powder having a vitamin E content of 72.5%. The resultant powder had good flowability. The bulk density of the powder was 3.27 ml/g.

Spray drying conditions: inlet temperature 220° C., outlet temperature 105° C., disk revolution speed 30,000 rpm, and spray speed 2.0 L/hr.

### Example 15

Dissolved in 150 g of purified water heated to 50° C. were 20 g of gelatin and 2.0 g of sodium carboxymethyl cellulose (the name of a maker: Daicel Chemical Industries, Ltd.), and 100 g of dl-α-tocopherol acetate (vitamin E) were added to the solution. The resultant mixture was stirred for 10 minutes by a Homomixer (trade name). Further, 1,650 g of purified water heated to about 50° C. were added to the mixture, followed by stirring for 10 minutes by the Homomixer. To the resultant mixture were added 80 g of anhydrous silicic acid (Aerosil 389), and the resultant mixture was stirred for 15 minutes by the homomixer to obtain a homogeneous suspension. The viscosity of the suspension was 40 cp. The suspension was spray-dried under the following conditions to obtain 118 g of flowable powder having a vitamin E content of 50%. The resultant powder had good flowability. The bulk density of the powder was 2.70 ml/g.

Spray drying conditions: inlet temperature 220° C., outlet temperature 105° C., disk revolution speed 30,000 rpm, and spray speed 2.0 L/hr.

### Example 16

Dissolved in 160 g of purified water heated to 50° C. were 20 g of casein and 2.0 g of sodium carboxymethyl cellulose (the name of a maker: Daicel Chemical Industries, Ltd.), and 80 g of dl-α-tocopherol acetate (vitamin E) were added to the solution. The resultant mixture was 5 stirred for 10 minutes by a Homomixer (trade name). Further, 300 g of purified water heated to about 50° C. were added to the mixture, followed by stirring for 10 minutes by the Homomixer. To the resultant mixture were added 58 g of anhydrous silicic acid (Aerosil 380, trade name), and the resultant mixture was stirred for 15 minutes by the homomixer to obtain a homogeneous suspension. The viscosity of the suspension was 128 cp. The suspension was spray-dried under the following conditions to obtain 116.6 g (yield: 72.9%) of flowable powder having a vitamin E content of 50%. The resultant powder had good flowability. The bulk density of the powder was 2.48 ml/g.

Spray drying conditions: inlet temperature 220° C., outlet temperature 105° C., disk revolution speed 30,000 rpm, and spray speed 1.5 L/hr.

### Example 17

Dissolved in 60 L of water were 2.5 kg of hydroxypropyl methylcellulose and 5.0 kg of gelatin at about 50° C. To this solution were added 30 kg of dl-α-tocopherol acetate (vitamin E) under stirring, and the resultant mixture was subjected to an emulsifying treatment by a Homomixer (trade name). Then, 45 L of water were added to the resultant emulsion, and the resultant mixture was subjected again to the emulsifying treatment. To the resultant suspension were added 7.5 kg of porous powder of calcium silicate (product of Tokuyama Soda Co., Ltd., trade name: Florite RE) with stirring. The resultant suspension was spray-dried under conditions of an inlet temperature of 230° C., an outlet temperature of 135° C. to 140° C. and a feed flow rate of 105 kg/hr to obtain 37.2 kg (yield: 82.6%) of white flowable powder having a vitamin E content of 67%.

The particle diameter distribution of the resultant powder was as follows: 60 mesh (hereinafter referred to as M) (15.2%), 100 M (64.1%) and 200 M (18.1%), and no aggregation among powder particles was observed.

### Example 18

Dissolved in 60 L of water were 2.5 kg of hydroxypropyl methylcellulose and 5.0 kg of gelatin at about 50° C. To this solution were added 30 kg of dl-α-tocopherol acetate (vitamin E) under stirring, and the resultant mixture was subjected to an emulsifying treatment by a Homomixer (trade name). Then, 50 L of water were added to the resultant emulsion, and the resultant mixture was subjected again to the emulsifying treatment. To the resultant suspension were added 7.5 kg of Florite RE with stirring. The resultant suspension was spray-dried under conditions of an inlet temperature of 211° C. to 219° C., an outlet temperature of 132° C. to 134° C. and a feed flow rate of 95 to 105 kg/hr to obtain 36.4 kg (yield: 80.9%) of white flowable powder having a vitamin E content of 67%.

The particle diameter distribution of the resultant powder was as follows: 60 M (24.5%), 100 M (56.3%) and 200 M (16.3%), and no aggregation among powder particles was observed.

### Example 19

A process was carried out in the same manner as in Example 16 except that 10 g of casein and 10 g of gelatin were used in place of 20 g of casein. The results thereof were as follows:

153 g (yield: 76.5%) of flowable powder having a vitamin E content of 50%; bulk density of the powder: 2.35 ml/g.

### Example 20

A process was carried out in the same manner as in Example 1 except that 120 g of teprenone were used in place of 120 g of dl-α-tocopherol acetate. The results thereof were as follows:

136 g (yield: 68%) of flowable powder having a teprenone content of 60%; bulk density of the powder: 4.2 ml/g.

1. Flowable powder comprising (A) a fat-soluble drug, (B) at least one selected from the group consisting of gelatin and casein, and (C) an adsorbent.

2. Flowable powder comprising (A) a fat-soluble drug, (B) at least one selected from the group consisting of gelatin and casein, (D) a water-soluble polymer, and (C) an adsorbent.

3. The flowable powder according to claim 1 or 2, wherein the fat-soluble drug is a fat-soluble drug having a melting point of 80° C. or lower.

4. The flowable powder according to any one of claims 1 to 3, wherein the fat-soluble drug is one of vitamin E family.

5. The flowable powder according to claim 1, which comprises the fat-soluble drug, gelatin and the adsorbent.

6. The flowable powder according to claim 1, which comprises the fat-soluble drug, casein and the adsorbent.

7. The flowable powder according to claim 1, which comprises the fat-soluble drug, gelatin, casein and the adsorbent.

8. The flowable powder according to any one of claims 1, 2 and 5 to 7, wherein the adsorbent is a silicate.

9. The flowable powder according to claim 8, wherein the silicate is one or more selected from the group consisting of silicon dioxide, calcium silicate, magnesium alumino silicate and magnesium alumino metasilicate.

10. The flowable powder according to any one of claims 1, 2, 8 and 9, wherein the adsorbent is a porous inorganic substance.

11. The flowable powder according to claim 2, wherein the water-soluble polymer is one or more selected from among cellulose derivatives and polyvinyl alcohol.

12. The flowable powder according to claim 1, which comprises, per part by weight of the fat-soluble drug, 0.05 to 0.5 parts by weight of gelatin and/or casein, and 0.1 to 0.8 part by weight of the adsorbent.

13. The flowable powder according to claim 2, which comprises, per part by weight of the fat-soluble drug, 0.05 to 0.5 parts by weight of gelatin and/or casein, 0.1 to 0.7 part by weight of the adsorbent, and 0.01 to 0.1 parts by weight of the water-soluble polymer.

14. A process for producing flowable powder, which comprises emulsifying and dispersing (A) a fat-soluble drug, (B) gelatin and/or casein, arid (C) an adsorbent, and optionally (D) a water-soluble polymer in water and spray-drying the resultant dispersion.
